(19) United States
(12) Patent Application Publication
(10) Pub. No.: US 2009/0022693 A1
(43) Pub. Date: Jan. 22, 2009

(54) METHOD FORISOLATING STEM CELLS FROM CRYOPRESERVED DENTAL TISSUE

(75) Inventors: Michael Thie, Bonn (DE); Ozer Degistirici, Bonn (DE)

Correspondence Address: JOYCE VON NATZMER;PEQUIGNOT + MYERS LLC, 200 Madison Avenue, Suite 1901, New York, NY, 10016, US

(73) Assignee: STIFTUNG CAESAR, Bonn (DE)

(21) Appl. No.: 11/997,194

(22) PCT Filed: Jul. 17, 2006
(86) PCT No.: PCT/EP2006/006995

§ 371 Date: Sep. 30, 2008

(30) Foreign Application Priority Data
Jul. 29, 2005 (EP) 05016549.7
Jun. 27, 2006 (DE) 10 2006 029 832.2

(51) Int. Cl. A61K 35/12; C12N 5/08; A61P 43/00

(52) U.S. Cl. 424/93.7; 435/381; 435/368; 435/366

(57) ABSTRACT

The invention relates to a method for isolation of multipotent stem cells from dental tissue in which the stem cells are extracted from a tissue structure and then cultured. The invention also relates to stem cells isolated by means of the method according to the invention as well as bone cells and nerve cells produced by means of the method according to the invention. The invention also relates to a method for producing a bank of stem cells in which the cells are stored by means of the method according to the invention. According to the present invention, the cells of a pad-like soft tissue that can be localized beneath the papilla directly on the apical side of an extracted immature tooth are cryopreserved in the tissue structure such that the tissue structure is disintegrated to extract the stem cells only after thawing. These results show that stem cells/progenitor cells can be isolated even after cryopreservation of the source tissue and these stem cells/progenitor cells respond to osteogenic stimulation. In addition, the response of cells after cryopreservation turns out to be stronger than that without cryopreservation.

### BACKGROUND OF THE INVENTION

The present invention relates to a method for isolating multipotent stem cells from dental tissue in which the stem cells are extracted from a tissue structure and then cultured. The invention also relates to stem cells as well as bone cells and nerve cells isolated and prepared by the method according to the invention. The invention also relates to a method for producing a stem cell bank in which the cells are stored by the method according to the invention.

Stem cells are somatic cells that are not differentiated, i.e., stem cells are not yet specialized for a task in the body, e.g., as skin cells or liver cells. Stem cells may be formed by division of other stem cells and/or may originate from differentiated cells, i.e., stem cells are also capable of asymmetric division. A stem cell retains its ability to divide over a very long period of time often even during the entire lifetime of the body. Triggered by specific signals during the development of the body, a stem cell may differentiate into different types of cells, which then form the body. A distinction is made in general between embryonal stem cells and adult stem cells.

Embryonal stem cells (ES cells) from which an embryo develops up to the eight-cell stage are referred to as totipotent. All cell forms of the developing body subsequently develop from these cells. Embryonal stem cells from the blastocyst stages are known as pluripotent cells because all types of body cells of the main tissue types can usually be differentiated from them, namely endoderm (wall cells of the digestive tract), mesoderm (muscles, bones, blood cells) and ectoderm (skin cells and nerve tissue). However, for ethical reasons and because of problems with molecular control of cell differentiation, ES cells have not previously been therapeutically usable.

Adult stem cells (AS cells) however are formed after the embryonal stage, i.e., they are undifferentiated cells which accumulate in a differentiated tissue and from which arise specialized cells corresponding to those of the differentiated tissue. However, ES cells may also differentiate into cell types that are to be assigned to a different tissue. However, adult stem cells that can be found in organs, in bone marrow or in the umbilical cord can no longer differentiate as freely as embryonal stem cells. Although adult stem cells do not have the same differentiation potential as embryonal stem cells, they nevertheless have a differentiation potential exceeding that of the ectoderm stage. They are therefore referred to as multipotent. For example, mesenchymal stem cells can also differentiate to nerve cells, which otherwise develop from ectodermal tissue. Thus, after accumulating in a different type of tissue, AS cells are capable of differentiating into a cell type that does not correspond to the cell type of their parent tissue. Adult stem cells are available in each individual, e.g., in the bone marrow. However, extraction of bone marrow is a complex and risky surgical procedure. In contrast with that, obtaining stem cells from dental tissue is a less complex alternative, as described in WO 03/066840 A2, for example, for AS cells from dental follicles. Such stem cells from readily accessible tissue open up the prospect of tissue replacement by endogenous cells, for example. The tendency to malignancy after implantation of adult stem cells also appears to be lower than with embryonal stem cells. Adult stem cells are thus of growing importance for the development of innovative therapeutic approaches.

The term cryopreservation is understood to refer to freezing and storage of biological material such as live cells or tissues in or above liquid nitrogen, i.e., at temperatures below −130° C. The temperature of liquid nitrogen is −196° C., but nitrogen enters the gaseous physical state at higher temperatures under normal pressure. By freezing the cells at such low temperatures, the essential biological functions of the cells come to a standstill, so that long-term storage is possible with little or no damage to the material. Special cryopreservation methods are used, in which the cells are placed in a cell membrane protective medium (cryoprotective) and are frozen using specific computer-controlled temperature programs. Cryopreservation is often used in in-vitro and other fertility treatments by freezing and storing sperm or fertilized egg cells. However, stem cells can also be stored by cryopreservation.

The known methods of cryopreservation of whole teeth have the disadvantage that cell death and drastic cell loss in the tissue occur and therefore the vitality rate of cells after thawing is very low.

### STATE OF THE ART

WO 2005/052140 A2, for example, discloses a method for cryopreservation of dental tissue from which stem cells can be isolated after thawing. The alveolar periostium (=periodontal ligament) is frozen and thawed in an uncritical manner, i.e., without a controlled procedure, as the partial tissue of a tooth to be preserved. Serum containing 1% to 20% dimethyl sulfoxide (DMSO) is proposed as a cryoprotective medium. The tissue is placed in the cryoprotective medium and flash-frozen in liquid nitrogen. Frozen and stored tissue is then thawed again at 35-39° C. However, this known method results in a high cell loss in the tissue, and the few cells that can be isolated also have a very low stem cell colony rate (Seo et al., 2005).

Papaccio et al. (2006) discovered that isolated stem cells from dental pulp (pulpa dentis), even after being in storage for two years, do not lose their stem cell properties due to cryopreservation and can still be differentiated into bone cells. However, using the method described in WO 2005/052140 A2 (Seo et al., 2005), no stem cells at all could be isolated from cryopreserved dental pulp tissue.

### SUMMARY OF THE INVENTION

The object of the present invention is to provide a method for isolating stem cells from dental tissue that will ensure a high yield of multipotent adult stem cells.

This object is achieved according to this invention by a method for isolating multipotent stem cells of the type defined in the introduction in which the cells of a pad-like soft tissue, which can be localized beneath the papilla directly on the apical side of an extracted immature tooth, are cryopreserved in the tissue structure, which is disintegrated to extract the stem cells only after thawing. This inventive method advantageously allows isolation of multipotent ectomesenchymal stem cells/progenitor cells from special dental tissue (apical pad) from which ectomesenchymal stem cells/progenitor cells can be isolated especially easily. It has surprisingly been found that even after cryopreservation of this pad-like soft tissue, stem cells and/or precursor cells can still be isolated from the united tissue structure. The response of the cells to osteogenic stimulation is surprisingly even higher after cryopreservation than without the intermediate step of cryopreservation. Cryopreservation thus serves practically as a stimulus for the ability of the isolated stem cells to differentiate. The multipotency of the stem cells thereby isolated is evidently stimulated, i.e., optimized by the method according to the invention and in particular by cryopreservation. The tissue selected as the source of the stem cells, e.g., obtained in extraction of wisdom teeth, also opens up the possibility of having access to (autologous) source tissue containing stem cells even in the future, assuming they are stored. The method according to the invention in particular holds open the possibility of still having access to this total population of all cells in the source tissue as part of a cell replacement therapy, if isolation protocols are established for special cell populations. This yields the advantage of long-term storage and rapid availability of highly potent tissue for subsequent therapeutic purposes as needed.

In an advantageous embodiment of the method according to the invention, the cells of the tissue structure are cooled in a controlled manner in a freezing medium for cryopreservation in such a way that the formation of intracellular ice begins at a temperature of approximately −7 to −12° C., preferably −10° C., and after the ice has formed, the cells are cooled further down to a temperature of at most −80° C. and are stored in or above liquid nitrogen. Due to the controlled freezing, the cells are protected and thus the yield of viable stem cells is increased. In a particularly advantageous embodiment of the invention, the cells are cooled in such a way that the formation of ice begins after 20-25 minutes, preferably 25-30 minutes, especially 27-29 minutes. Due to the choice of the point in time of intracellular ice formation, the method according to the invention can be adapted to individual types of cells and/or types of tissue and can be further optimized with regard to yield.

Surprisingly the method according to the invention can also be further optimized by triggering the formation of ice by controlled use of a seed crystal. In this embodiment, the cryoprotective medium together with the tissue to be frozen is cooled down to a temperature of −10 to −12° C. and then a seed crystal can be used by touching the vessel with an object from the outside, leading to abrupt freezing of the cryoprotective medium. This procedure has the advantage that the point in time of formation of ice can be selected in a controlled manner and furthermore the location of the start of ice formation, preferably near the tissue to be frozen, can be influenced. In this way the stress to which the cells are exposed during the freezing process is greatly reduced, and this is in turn manifested in a further increase in the yield of viable stem cells.

After the ice has formed, the cells of the tissue structure can then be cooled down to a temperature between −90° C. and −160° C., preferably between −100° C. and −150° C., in particular between −120° C. and −130° C., for permanent storage, and then after cryopreservation, the cells can be thawed by heating to 35-39° C.

With regard to the survival rate of the cells, it has been found to be especially advantageous if the cells of the tissue structure are thawed in several steps by dilution of the freezing medium. The freezing medium may be replaced incrementally, e.g., with a medium containing 50%, 25%, 12.5%, 6.25% and 0% fetal calf serum (FCS). The survival rate of the stem cells can be further increased by this gradual thawing.

In an especially advantageous embodiment of the method according to the invention, the freezing medium comprises a salt solution, preferably PBS, with 10 mg/mL serum albumin, 0.1 M sucrose and 1.5 M PrOH. The composition of the cryoprotective medium may of course be adapted to the respective tissue to be frozen and thus the method according to the invention can be further optimized.

With regard to the multipotency of the stem cells to be isolated by the method according to the invention, it is especially advantageous if the pad-like soft tissue is obtained from an anlage of an impacted and/or retinated tooth in the development phase between the occurrence of the bony alveolar fundus and conclusion of the root formation. To be able to isolate these desired multipotent stem cells, the pad-like soft tissue should be separated from the tooth after surgical extraction of the tooth along a macroscopically visible border between the pad-like soft tissue and the papilla, preferably within an imaginary line between the developing root protrusions. The tissue selected in this way allows isolation of ectomesenchymal stem cells, i.e., precursor cells, which can be differentiated into various cell types, e.g., bone cells or nerve cells, because of their multipotency. The choice of the correct source tissue for isolation of the stem cells according to the inventive method is thus an important factor in obtaining a high yield of multipotent stem cells.

After thawing, the tissue structure can be disintegrated by enzymatic treatment, preferably with collagenase/dispase. In doing so, the cells can also be isolated after being extracted from the tissue structure.

The cells isolated by the method according to the invention are ectomesenchymal stem cells and/or precursor cells which can be stimulated osteogenically and/or neurogenically after being isolated from the tissue structure.

The invention also relates to bone cells and nerve cells that have been isolated by the method according to the invention. The stem cells isolated by the method according to the invention are also the subject matter of the present invention.

Due to their multipotency, the stem cells according to the invention are suitable in particular for therapeutic purposes within the context of a cell and/or tissue replacement therapy. Thus the method according to the invention maintains the option of access to the total population of all cells in the source tissue. This yields the advantage of long-term storage and rapid availability of highly potent tissue for subsequent therapeutic purposes as needed. To this end, a method for producing a bank of stem cells is provided, in which the cells are stored by means of the method according to the invention, wherein the pad-like soft tissue of a plurality of teeth can be cryopreserved and cataloged separately to select and isolate certain stem cells in a targeted manner as needed. The present invention also relates to a stem cell bank produced by this method.

This invention will now be explained in greater detail below on the basis of the figures and the exemplary embodiments as examples.

### BRIEF DESCRIPTION OF THE FIGURES

In the figures

FIG. 1 shows a perspective view of an extracted wisdom tooth with the apical soft tissue (apical pad),

FIG. 2 shows illustrations of the displays on a monitor showing the course of temperature during control freezing of the tissue according to the present invention, with
a) spontaneous formation of ice and
b) stimulated formation of ice,

FIG. 3 shows a micrograph of a cell colony with fibroblastoid cells that have been frozen and thawed according to the inventive method,

FIG. 4 shows a comparison of the proliferation behavior of samples treated in different ways (PK I: DMSO with a rapid thawing method; PK II: DMSO with a slow thawing method (dilution); PK III: sucrose with a rapid thawing method; PK IV: sucrose with a slow thawing method (dilution)); F=fresh, non-cryopreserved cells; N2=cryopreserved cells, a) table, b) bar graph,

FIG. 5 shows the result of a FACS analysis of the samples treated in different ways (PK I-PK IV), positive control: human bone marrow stem cells (hBMSC), and

FIG. 6 shows bar graphs to differentiate the stem cells/progenitor cells according to the invention after 21 days with and without osteogenic stimulation, negative controls: fibroblasts (EU2A), positive controls: human stem cells from bone marrow (hBMSC).

### DESCRIPTION OF VARIOUS AND PREFERRED EMBODIMENTS OF THE INVENTION

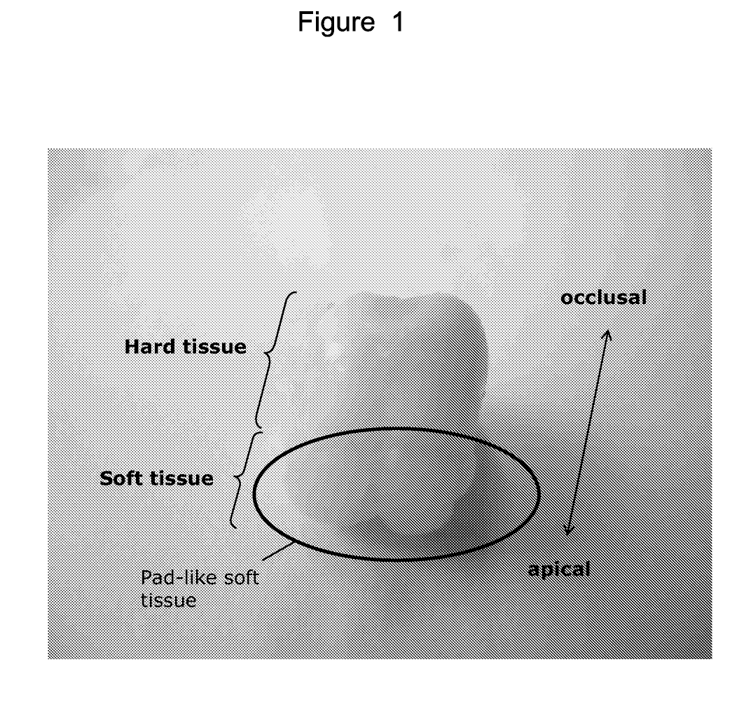

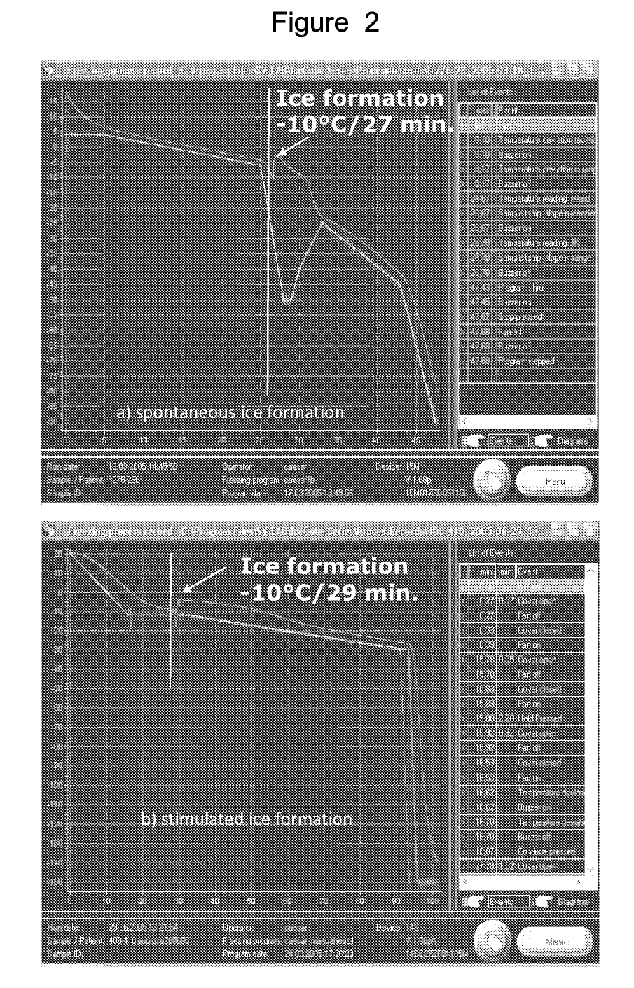

FIG. 1 shows an extracted wisdom tooth having a pad-like soft tissue on its apical side, that is placed as a dental tissue compartment to be frozen in a freezing medium (cryoprotective medium, a mixture of medium, 10% FCS and 10% DMSO or a mixture of PBS, serum albumin, sucrose and PrOH) and then is frozen under controlled conditions in an automatic freezer (IceCube) under set freezing parameters (cooling rate). The frozen samples are stored for longer periods of time at −196° C. (above liquid nitrogen). Thawing of the tissue at 37° C. is also critical and is performed either rapidly or slowly with incremental replacement of the cryoprotective medium with a normal medium (freezing medium containing 50%, 25%, 12.5%, 6.25% and 0% FCS). After thawing, the tissue is digested with collagenase/dispase by analogy with the fresh tissue. The isolated cells are cultured at 37° C. in DMEM+10% FCS and evaluated according to parameters such as vitality, proliferation ability, expression of surface markers and differentiation potential (including osteogenesis).

The method according to the invention for isolating multipotent stem cells by cryopreservation of viable dental tissue comprises the apical pad-like soft tissue as the tissue of a wisdom tooth to be frozen, two solutions suitable as the cryoprotective medium for this tissue, an adapted freezing step, an apparatus (IceCube) that automatically performs the freezing, a thawing step which is also adapted and a selection of features with the help of which the quality of the tissue can be checked practically after thawing.

The extracted wisdom tooth, i.e., taken surgically from a person according to FIG. 1 is placed in a container (tooth box) filled with transport medium (DMEM+penicillin+streptomycin) at room temperature in a cell culture laboratory. The apical pad (pad-like tissue) is separated from the tooth below the imaginary line between the root of the tooth and the tooth along the microscopically visible boundary between the pad-like soft tissue and the papilla, then washed several times with PBS (sterile) and next macerated with a scalpel. Half of the tissue preparation (N2) is mixed with freezing medium (a mixture of DMEM, 10% FCS and 10% DMSO or a mixture of PBS, serum albumin, sucrose and PrOH) and precooled in a controlled manner with a computer-controlled freezing unit and then frozen. In this exemplary embodiment, ice is formed after 27-29 minutes at a temperature of approximately −10° C. either spontaneous due to extreme cooling, i.e., introduction of liquid nitrogen into the cooling chamber or through controlled use of a seed crystal in the freezing vessel. In the case of the latter embodiment, a seed crystal that leads to sudden freezing of the cryoprotective medium is placed in the cryoprotective medium with the tissue to be frozen by bringing the surface of the container in contact with a precooled metal, leading to a sudden freezing of the cryoprotective medium. The start of formation of ice is indicated by the release of latent heat in the specimen container. FIG. 2 shows the temperature curve (cooling rate) during controlled freezing of the tissue by the method according to this invention. After the ice has formed, the sample is cooled further and is stored over liquid nitrogen on reaching a temperature of −90° C. and/or −150° C. After storage, the tissue is heated to 37° C. either in a one-step rapid thawing process or in several slow steps (first freezing medium diluted with 50% FCS, then 25%, 12.5%, 6.25% and 0%). After digestion of the tissue with collagenase/dispase for two hours at 37° C., the isolated cells are washed several times and then cultured in 10% FCS+DMEM (LG contained T25 bottles). The medium was replaced every third to fourth day. The second half of the tissue preparation (F, see above) is processed directly without having to run through the previous freezing operation.

The cells isolated from this tissue were then analyzed according to various criteria:
1. Duration of cell layer formation to confluence
2. Cell count after seven days
3. Analysis of surface markers
4. Osteogenic differentiability of the isolated stem cells/progenitor cells.

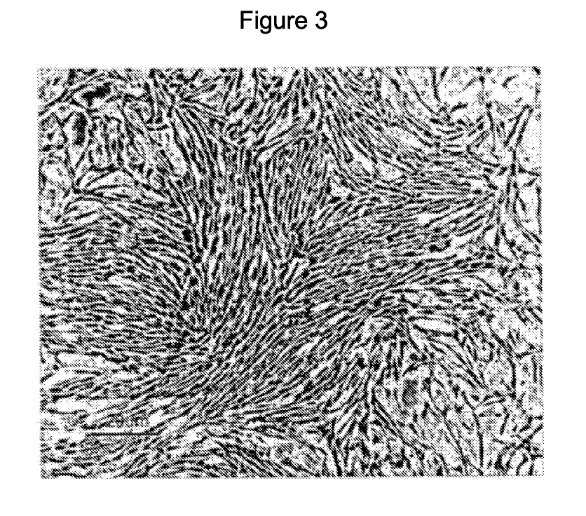

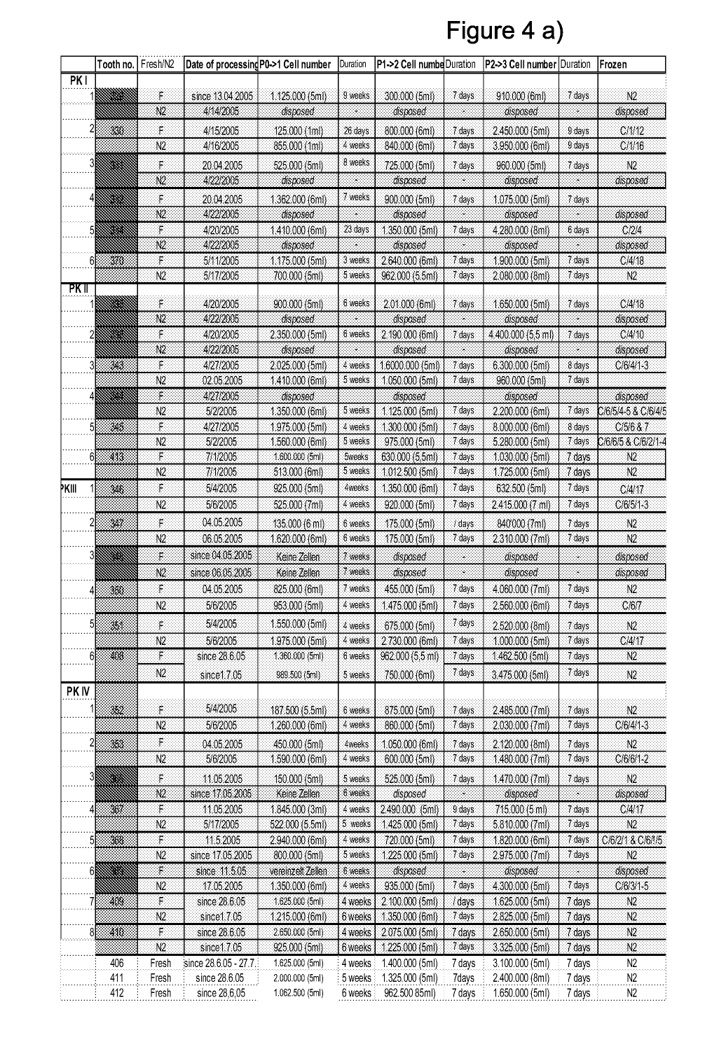

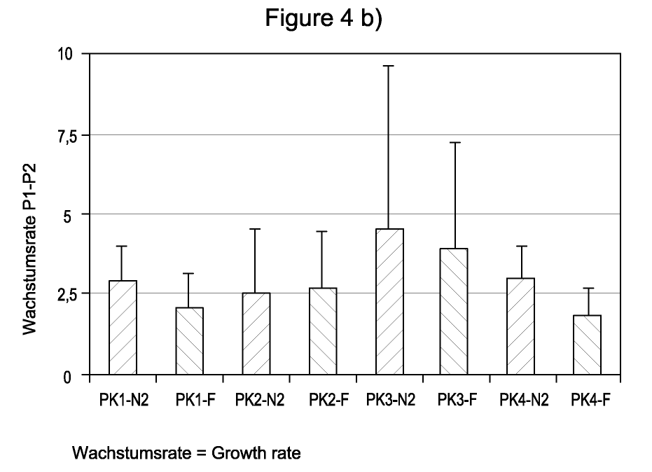

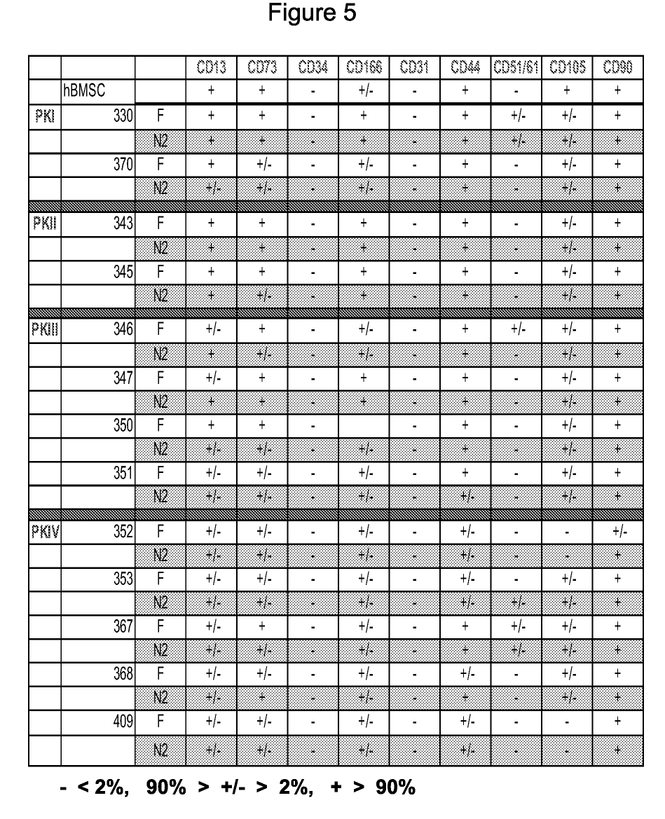

In culturing the cells from cryopreserved tissue, the first colonies are observed after one to three weeks (FIG. 3). Differences between the protocol variants (PKI: DMSO with a rapid thawing process; PKII: DMSO with a slow thawing process (dilution); PKIII: sucrose with a rapid thawing process; PKIV: sucrose with a slow thawing process (dilution)) are not significant. With regard to their proliferation behavior, the cells from cryopreserved tissue (N2) are comparable to cells from native material (F=fresh) in that the cells from cryopreserved tissue to some extent have even higher growth rates than fresh cells, which also indicates that cryopreservation at least does not damage the cells (FIG. 4). The analysis of the surface markers of the isolated cells also shows only minor differences in the expression pattern between cryopreserved and native source tissue (FIG. 5).

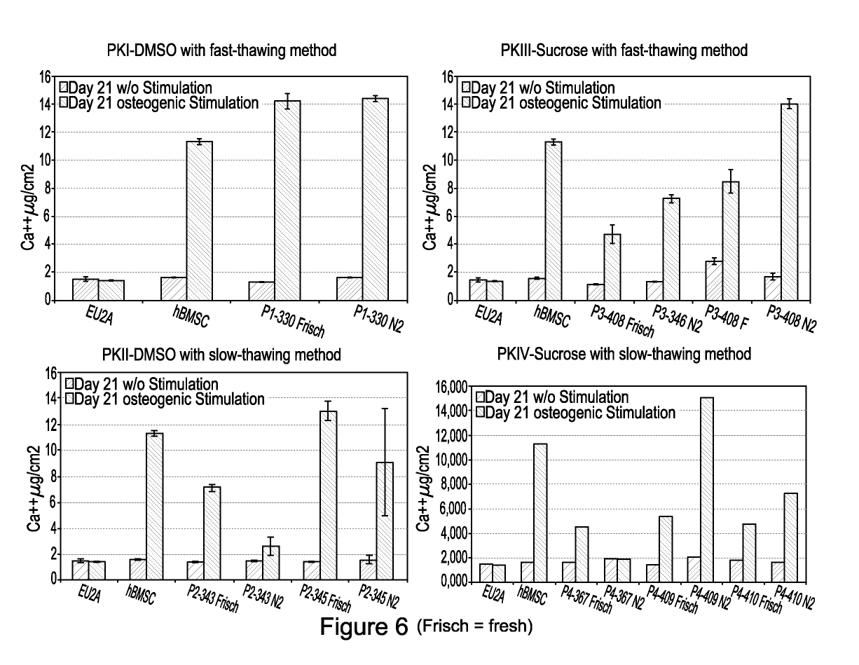

FIG. 6 shows bar graphs of the osteogenic differentiation ability of the cells after corresponding stimulation, where the degree of calcification was determined by determining the calcium ion concentration. These results show that even after cryopreservation of the source tissue, stem cells/progenitor cells can be isolated and these stem cells/progenitor cells respond to osteogenic stimulation. In addition, the response of the cells after cryopreservation surprisingly turns out to be even greater than that without cryopreservation. The protocol variant IV (PK IV) shows the highest osteogenic response of the isolated cells, i.e., the highest differentiation capacity and/or the highest yield of stem cells is obtained by using the cryopreservative medium containing sucrose in combination with the slow thawing method (thawing by dilution of the cryoprotective).

### REFERENCES

Seo B M, Miura M, Sonoyama W, Coppe C, Stanyon R, Shi S.: Recovery of stem cells from cryopreserved periodontal ligament; J Dent Res. 2005 October; 84(10):907-12
Papaccio G, Graziano A, d'Aquino R, Graziano M F, Pirozzi G, Menditti D, De Rosa A, Carinci F, Laino G: Long-term cryopreservation of dental pulp stem cells (SBP-DPSCs) and their differentiated osteoblasts: A cell source for tissue repair; Journal of cellular physiology Vol: 208 (2); 319-25, 2006

1. A method for isolation of multipotent stem cells from dental tissue in which the stem cells are extracted from a tissue structure and then cultured
comprising
cryopreserving in the tissue structure cells of a pad-like soft tissue, localized below the papilla directly on the apical side of an extracted immature tooth,
disintegrating the tissue structure to extract stem cells only after thawing, and
isolating multipotent stem cells.

2. The method according to claim 1,
wherein
the cells are cooled in a controlled manner in a freezing medium for cryopreservation in such a way that the formation of intracellular ice starts at a temperature of approximately −7° to −12° C., and the cells are cooled further to a temperature of maximally −80° C. after ice has formed and are stored in or above liquid nitrogen.

3. The method according to claim 2,
wherein
the cells are cooled in such a way that the formation of ice begins after 20-25 minutes.

4. The method according to claim 2,
wherein
the formation of ice is triggered by controlled use of a seed crystal.

5. The method according to claim 2,
wherein
after the ice is formed, the cells are cooled down to a temperature between −90° C. and −160° C.

6. The method according to claim 1,
wherein
after cryopreservation, the cells are thawed by heating to 35-39° C.

7. The method according to claim 6,
wherein
the cells are thawed in several steps by dilution of the freezing medium.

8. The method according to claim 7,
wherein
the freezing medium is replaced incrementally with a medium containing 50%, 25%, 12.5%, 6.25% and 0% fetal calf serum (FCS).

9. The method according to claim 2,
wherein
the freezing medium comprises a salt solution containing 10 mg/mL serum albumin, 0.1 M sucrose and 1.5 M PrOH.

10. The method according to claim 1,
wherein
the pad-like soft tissue is extracted from an anlage of an impacted and/or retinated tooth in a development phase between occurrence of the bony alveolar fundus and conclusion of root formation.

11. The method according to claim 1, wherein
after surgical extraction of the tooth, the pad-like soft tissue is separated from the tooth along a macroscopically visible border between the pad-like soft tissue and the papilla.

12. The method according to claim 1, wherein
the tissue structure is disintegrated by enzymatic treatment and/or the cells are isolated after extraction from the tissue structure.

13. The method according to claim 1, wherein
the cells are ectomesenchymal stem cells and/or precursor cells.

14. The method according to claim 1, wherein
the cells are stimulated osteogenically and/or neurogenically after being isolated from the tissue structure.

15. A bone cell isolated by the method according to claim 14.

16. A nerve cell isolated by the method according to claim 14.

17. A stem cell isolated by the method according to claim 1.

18. A method for cell and/or tissue replacement therapy comprising providing the stem cell of claim 17, wherein said stem cell is provided for therapeutic purposes within a context of said cell and/or tissue replacement therapy.

19. The method for producing a bank of stem cells in which the cells are stored by the method according to claim 2, wherein the pad-like soft tissue of a plurality of teeth is cryopreserved and cataloged separately to be able to select and isolate certain stem cells in a targeted manner as needed.

20. The stem cell bank produced by means of the method according to claim 19.

21. The method according to claim 2,
wherein said formation of intracellular ice starts at a temperature of −10° C.

22. The method according to claim 3,
wherein the cells are cooled in such a way that the formation of ice begins after 25-30 minutes.

23. The method according to claim 3,
wherein the cells are cooled in such a way that the formation of ice begins after 27-29 minutes.

24. The method according to claim 5,
wherein the cells are cooled down to a temperature between −100° C. and −150° C.

25. The method according to claim 24,
wherein the cells are cooled down to a temperature between −120° C. and −130° C.

26. The method according to claim 9,
wherein the salt solution is PBS.

27. The method according to claim 12,
wherein the tissue structure is disintegrated by enzymatic treatment with collagenase/dispase.
